(19) United States
(12) Patent Application Publication
(10) Pub. No.: US 2018/0072996 A1
(43) Pub. Date: Mar. 15, 2018

(54) Vascularized Skin Models

(71) Applicant: The Government of the United States of America, as represented by the Secretary of the Navy, Arlington, VA (US)

(72) Inventors: André A. Adams, Burke, VA (US); Kyle A. DiVito, Burkittsville, MD (US)

(21) Appl. No.: 15/698,752

(22) Filed: Sep. 8, 2017

Related U.S. Application Data
(60) Provisional application No. 62/385,302, filed on Sep. 9, 2016.

(51) Int. Cl. C12N 5/071; C12M 1/00

(52) U.S. Cl. CPC C12N 5/0698; C12M 29/10; C12N 2502/28; C12N 2502/091; C12N 2502/1323; C12N 2502/094; C12N 2502/1347

(57) ABSTRACT

A synthetic skin tissue is served by at least one synthetic blood vessel and includes a physiologically representative population of cells including fibroblasts and keratinocytes, wherein some keratinocytes are in a differentiated state.

### CROSS-REFERENCE TO RELATED APPLICATIONS

This Application claims the benefit of U.S. Provisional Application 62/385,302 filed on Sep. 9, 2017, the entirety of which is incorporated herein by reference.

This application is related to U.S. Pat. No. 9,157,060, incorporated herein by reference for disclosing synthetic blood vessels and tissue ducts and the methods of making them; and to US Patent Application Publication 2016/0130543, incorporated herein by reference for disclosing techniques for perfusing such synthetic vessels

### BACKGROUND

The skin is the largest organ of the human body and serves a critical protective role. Unlike other tissue types with discrete roles such as absorption or filtration as in the intestine or liver, respectively, the role of the skin is quite diverse. As the primary barrier to the environment, the skin's task is to protect internal organs from exposure to the elements. The elements can include airborne particulates, infection from bacterial and/or viral sources, and protection from the damaging effects from the sun's ultraviolet light (UV). The three integral cellular components that comprise human skin are keratinocytes, fibroblasts and melanocytes. Keratinocytes generate the epithelia, the uppermost layer of the skin (FIG. 1), and make up approximately 80% of the skin (Prost-Squarcioni 2006). Dermal fibroblasts are located in the dermal layer of the skin (FIG. 1) and serve as the main structural support for the epidermis, generating a scaffold to which the epithelium attaches. Dermal fibroblasts also play a connective-tissue role. Melanocytes reside at the dermal-epidermal junction (FIG. 1) and are responsible for melanosome production which ultimately gives skin its pigmentation. Melanosomes are tiny organelles present in melanocytes that are the sole source of melanin, the chemical responsible for protection from UV damage (Natarajan, Ganju et al. 2014; Yamaguchi and Hearing 2014). Tissue macrophages or Langerhans's cells are the primary immune cells involved in the initial immune response to invaders, such as bacteria. Tactile sensation (touch) is the responsibility of Merkel cells (Prost-Squarcioni 2006). Other skin appendages, such as eccrine and apocrine sweat glands, are quite numerous with ˜600/cm² serving to provide lubrication and hydration to the skin; hair follicles and sebaceous glands are also numerous in regions of human skin such as the axilla (armpit) and the peritoneum (mid-section) (Sonner, Wilder et al. 2015). These other cell types are found in significantly lower numbers within the skin than the primary cell types described above.

A pervasive vasculature serves the human skin and plays integral roles in sensory function, thermoregulation, and host defense (Sonksen 1999). Beginning with the lowermost layer of the skin and the region closest to the internal muscle, the hypodermis is composed mostly of subcutaneous fat (FIGS. 1, 2A). This region is where the larger blood vessels of the skin reside. Here relatively large diameter arterioles and venules (50-100 μm outer diameter) reside to supply the skin with nutrients and remove waste. The region of skin exposed to the environment, the epithelium, itself is avascular. Though the region just below the epidermis is called the superficial papillary dermis, here, branches of blood vessels derived from the larger arterioles and venules present in the hypodermis establish connections and allow blood to recirculate through the skin (FIG. 2B)

To date, tissue models of human skin fall into either of two general categories in vitro or in vivo. Each suffers from complicating factors that ultimately diminish the efficacy of their use.

Purely in vitro human skin reproductions are often referred to as ‘skin equivalents’ or ‘raft cultures’. These models were initially developed in the early 1980's to address wound healing, and then later refined by M. Herlyn's laboratory at University of Pennsylvania (Worst, Mackenzie et al. 1982; Roop, Lowy et al. 1986; Conway, Morgan et al. 1992; Berking and Herlyn 2001) for use in studies of skin cancer progression. Skin equivalents rely, first, on the in vitro purification of each cell type present in human skin and the establishment of independent in vitro cultures of those cell types. Cell-culture well plate inserts are used to generate skin equivalents and they have a porous membrane coated with extracellular matrix proteins onto which cells can be attached in order to mimic tissue, here the individual cell-types that make up the skin are added in stepwise fashion and cultured. Skin equivalents have advantages and disadvantages. In terms of advantages, they are easily controlled and provide a good research model for cancers and/or pharmacodynamics observations (Paolini, Orecchia et al. 2013). Though, disadvantages to this model are pervasive. The model is difficult to assemble, time consuming and contains only the major components of the skin such as keratinocytes, melanocytes and fibroblasts; skin appendages such as hair follicles and sebaceous glands are typically lacking. Further, the model is a short-term assay that requires continual monitoring and growth-media replacement making long-term studies such as chemical- or biological-agent transmission across the skin difficult to execute. Yet, the most critical feature lacking in this system is the presence of a functional vasculature, which therefore limits in vitro skin models to <1 mm thick. Even modern skin equivalents generated using induced pluripotent stem cells (iPSC) still lack an effective approach to integrate a functional vasculature (Itoh, Umegaki-Arao et al. 2013). Skin equivalents are simple models of skin that disregard integral information obtained when tissues are placed in concert with a functioning ‘organ unit’ such as skin along with its associated vasculature. Questions such as immune cell transport and infiltration; how changes in blood pH effect tissue environments; how systemic infection may transit through the vasculature and may terminate in the skin and vice versa are essential experiments that cannot be accomplished using skin equivalents.

In vivo skin reproductions can be assembled in one of two manners. Either individual human skin cells, grown in culture, are introduced to an immunocompromised animal using a xenograft approach (Juhasz, Albelda et al. 1993), or whole skin human explants are sutured onto a rodent model for growth; both cases result in a xenograft model (Worst, Valentine et al. 1974; Worst, Mackenzie et al. 1982). The sole advantages of this system over the skin equivalent model are the presence of vasculature and the ability to use a thicker skin construct. Here, the animal's blood supply will eventually (after >3-4 weeks) invade and potentially perfuse the xenograft, yet graft success rates are low especially in thick skin transplants. However, disadvantages are abundant. Arguably the most prevalent disadvantage to using this model is the animal to be grafted must be immune-compromised to avoid graft-versus-host rejection; meaning it cannot mount an immune response to the cellular material being grafted and integral features of the immune system such as B-cells, T-cells, natural killer and dendritic cells are not present. Another problematic reason in vivo skin models suffer is from the complementarity of the model. That is, while rodents can approximate the human they are not genetically identical and therefore conclusions drawn from mouse studies often do not translate to the human often confounding research endpoint conclusions. Furthermore, extensive restrictions for animal research have been implemented in the European Union since 2013 (Hartung, Blaauboer et al. 2011; von Aulock 2013), especially for skin cosmetics.

The above-noted restrictions offset the utility of current in vitro and in vivo skin models. Previously disclosed in vitro skin models are limited to thin skin, which is generated using the techniques previously described and rely only on passive diffusion of growth media, etc. to maintain those tissues. Providing a functional vasculature, in an in vitro setting, would lead to greatly enhanced capabilities for laboratory investigation. In the absence of an integrative vasculature laboratory skin models cannot accurately represent the conditions of normal skin using current in vitro models Accordingly, a need exists for models that can better approximate human.

### BRIEF SUMMARY

Synthetic human blood vessels called human endothelial microvessels (HEMV) can be constructed and integrated into a growing human skin tissue construct to generate a vascularized human skin model. A vascularized human skin model can dramatically increase the utility of in vitro skin constructs by providing the potential to address and model systemic responses associated with the skin such as bacterial/viral infection, nutrient delivery, waste removal, in-transit cancer cell migration, chemical/biological agent transmission through skin, skin oxygenation and studies related to thermoregulation.

In one embodiment, synthetic skin tissue includes a synthetic blood vessel in the form of a hollow tubule comprising a porous polymer and incorporating living endothelial cells and living smooth muscle cells within the vessel; living fibroblasts in a matrix surrounding the vessel; and living keratinocytes disposed atop the fibroblasts, wherein an upper layer of the keratinocytes is in a differentiated state with characteristics of corneocytes.

In a further embodiment, a vascularized skin construct includes a synthetic skin tissue comprising a synthetic blood vessel in the form of a hollow tubule comprising a porous polymer and incorporating living human endothelial cells and living smooth muscle cells within the vessel, living human fibroblasts in a matrix surrounding the vessel, and living human keratinocytes disposed atop the fibroblasts, wherein an upper layer of the keratinocytes is in a differentiated state with characteristics of corneocytes; and a perfusion manifold device comprising an inlet port and an outlet port configured to allow fluid flow through the vessel, the device having an opening exposing the the upper layer of keratinocytes to the atmosphere.

### BRIEF DESCRIPTION OF THE DRAWINGS

FIG. 1 illustrates a cross-section of human skin. The epidermis is the uppermost layer of the skin. The epidermis itself is then subdivided into 5 layers which include the: stratum corneum, stratum lucidum, stratum granulosum, stratum spinosum and stratum basale. The major cellular components of the epidermis are epidermal keratinocytes, epidermal melanocytes, as well as other secondary cell types, such as Langerhan's cells. The dermis is located just beneath the epidermis and acts as a support scaffold for the epidermis. The dermis is comprised of dermal fibroblasts and connective tissue. The hypodermis is composed of subcutaneous fat and is located beneath the dermal layer. Hair follicles, sebaceous glands and sweat glands are common skin appendages and they can be found in varying densities based upon skin location. The skin is highly vascularized with larger arterioles and venules originating in the hypodermis and branching off into smaller capillaries which extending upward into the upper papillary dermis. The epidermis itself is avascular.

FIGS. 2A and 2B illustrate the anatomy of vasculature in human skin. (A) Shows the outward extension of both arteriole and venule growth originating from the deep vascular plexus of the hypodermis into the papillary dermis. (B) Depicts anastomosis of neighboring blood vessels. Fusion of arteriole and venule blood vessels in capillary beds allows closed-loop circulation of the blood being delivered from the heart to surrounding tissue and back. Adopted from Sonksen, J. and Craggs, J. Circulation of the Skin. Current Anesthesia and Critical Care. (1999) 10, 58-63.

FIG. 3 is a schematic illustration of the formation of a human endothelial vessel incorporating endothelial cells, smooth muscle cells, and fibroblasts, all of human origin.

FIGS. 4A and 4B illustrate the growth of in vitro vascularized human skin using microfabricated blood vessels. FIG. 4A is a schematic depicting the placement of synthetic blood vessels into a human skin construct. Blood vessels are positioned into the dermal region of the skin containing dermal fibroblasts; human keratinocytes are then overlaid. After integration, the blood vessels undergo angiogenesis and the tissue construct grows together as an organ system. The image in FIG. 4B shows a synthetic blood vessel undergoing angiogenesis where new blood vessels are able to sprout off the original. Dermal fibroblasts present in the tissue surrounding the blood vessel are stained with DAPI (4′,6-diamidino-2-phenylindole) to identify nuclei. The scale bar is 100 μm long.

FIGS. 5A and 5B are images where anti-CD31 antibody is used to identify endothelial cells present within the blood vessel construct. Once embedded into the skin tissue constructs, the constructed blood vessels undergo angiogenesis and new blood vessels can permeate the surrounding tissue. Arrowheads show branch points where adjacent new-growth blood vessels are able to connect. The scale bar bars are 100 μm (A) and 50 μm (B) long.

FIGS. 6A and 6B illustrate a perfusion manifold device. In FIG. 6A, the top glass plate has a cut-away exposing the center cavity of the tissue construct. Epidermal exposure to air allows for air-liquid interface to become established and differentiation of the epidermis to generate the stratum corneum. FIG. 6B illustrates the assembly of manifold device, with the glass plate is placed over manifold and seals the system leaving only the center cavity of the vascularized skin construct exposed.

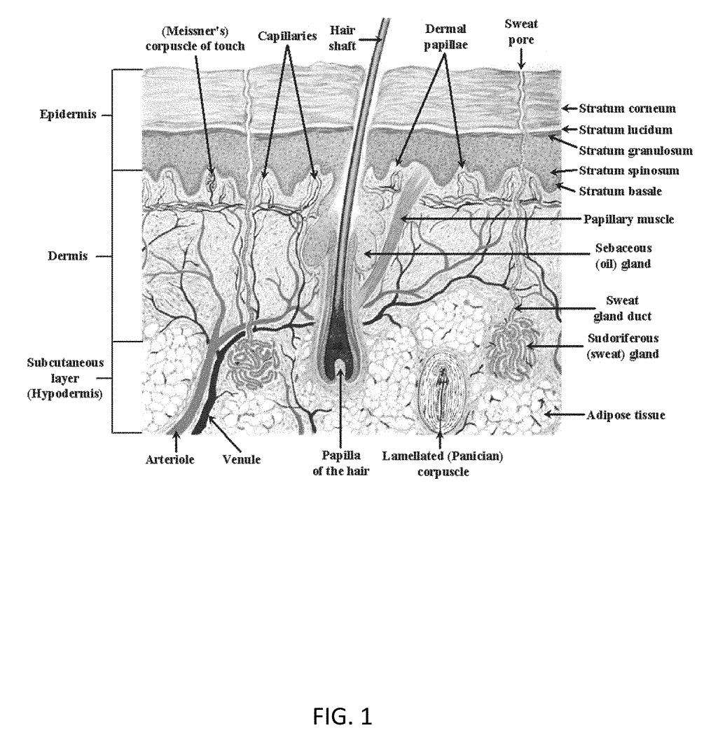

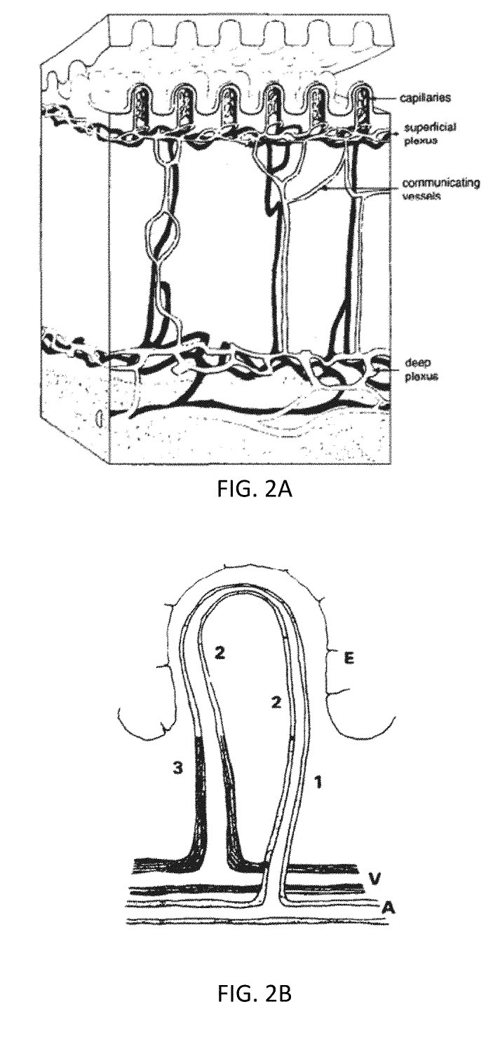

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

Overview

Previously disclosed in vitro skin models are limited to thin skin, which is generated using the techniques previously described and rely only on passive diffusion of growth media, etc. to maintain those tissues. Providing a functional vasculature, in an in vitro setting, would lead to greatly enhanced capabilities for laboratory investigation. In the absence of an integrative vasculature laboratory skin models cannot accurately represent the conditions of normal skin using current in vitro models.

Work using organ-on-a-chip devices or other tissue reconstructions have shown in vitro or in vivo tissue cannot be maintained beyond distances greater than ˜1 mm from the nutrient source (Tian and George 2011; Ehsan and George 2013). This is due to the diffusion limits of growth media and other nutrients. Systems that begin to recapitulate living tissue will need to either effectively model tissue vasculature such that blood vessels can be incorporated into such tissue models, otherwise the models will continue to be limited to thin tissue constructs which have been languishing for decades due to the inability to establish adequate perfusion. This is also true for in vitro skin equivalents, as the skin models generated are limited to ˜1 mm of thickness due to diffusion limits. However, regions of skin such as the thigh, soles of the feet and palms of the hand typically have skin depths of 3-5 mm. For this reason, tissue transmission studies using skin equivalents are limited to thin skin. The introduction of a perfusable vasculature would permit more complex studies capable of yielding a more complete picture of in vivo conditions, if tissue thickness issues were resolved via perfusion. This would significantly invigorate biomedical research, fortifying the toolkit available to addresses more complex questions allowing investigators to move beyond the limitations of the current state of the art.

Efforts have been made to incorporate tissue conduits or micro-channels integrated in between live tissue at distances nearing the diffusion limits (Kang, Lee et al. 2016). These conduits can be used to ‘perfuse’ the living material in order to maintain viability and to generate clinically relevant, size, shape and structural integrity of the tissue being modeled. However, what are overlooked using approaches such as these is that integrated microchannels do not reflect normal vasculature and lack essential cellular components of a blood vessel, mainly endothelial and smooth muscle cells. Blood vessels provide more than simply a conduit for which to transport material. The blood vessel itself is a dynamic system capable of contracting or dilating in response to given cues as well as allowing passive diffusion across the endothelial cell layer for certain molecules, while restricting others (Adams and Alitalo 2007). Further, blood vessels and, more specifically endothelial cells, are capable of considerable response to feedback from tissue. For example, in response to tissue injury or hypoxia, endothelial progenitor cells begin to organize and proliferate, branching off from larger vessels and thus generating a network of smaller vessels to help restore oxygenation and perfusion to the site of injury or hypoxia. This is a basic property of a properly functioning tissue system, yet rigid microchannels do not support angiogenesis and are incapable of responding to such cues limiting the thickness and efficacy of the resultant model.

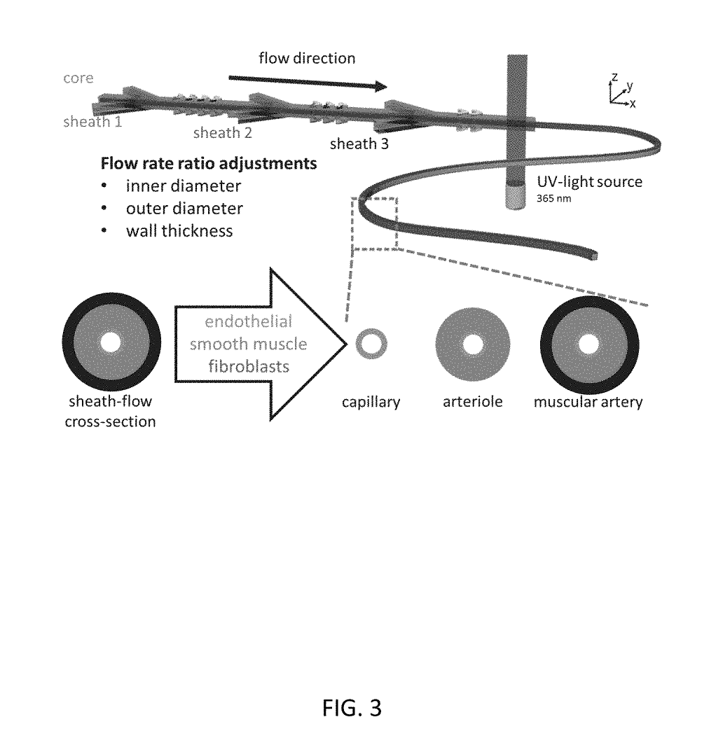

As described herein, vascularized human skin can better recapitulate the functional aspects of human skin architecture by providing tissue not only with perfusion but also the dynamic ability to respond to micro-environmental cues and respond accordingly. For example, one embodiment of a vascularized skin construct (VSC) will incorporate synthetic blood vessels to support the growth and maintenance of human skin for long term experimentation. It employs technology recently developed and patented (U.S. Pat. No. 9,157,060) at the U.S. Naval Research Laboratory to construct synthetic blood vessels, termed human endothelial microvessels (HEMV). Typically, a hollow core includes one or more concentric layers which can be populated with various cell types. FIG. 3 is a schematic illustration an exemplary technique for the formation of such vessels, showing how it might be scaled while including various cell types. Further details regarding the formation of such synthetic micro blood vessels and other fibers can be fond in U.S. Pat. Nos. 8,361,413, 8,398,935, and 9,573,311 as well as in M. Daniele et al., “Microfluidic fabrication of multiaxial microvessels via hydrodynamic shaping” RSC Adv., 2014, 4, 23440-23446. Each of these four patents and the journal article is incorporated herein by reference for the purposes of disclosing devices and methods (such as sheath flow) for preparing hollow fibers suitable for use as synthetic blood vessels.

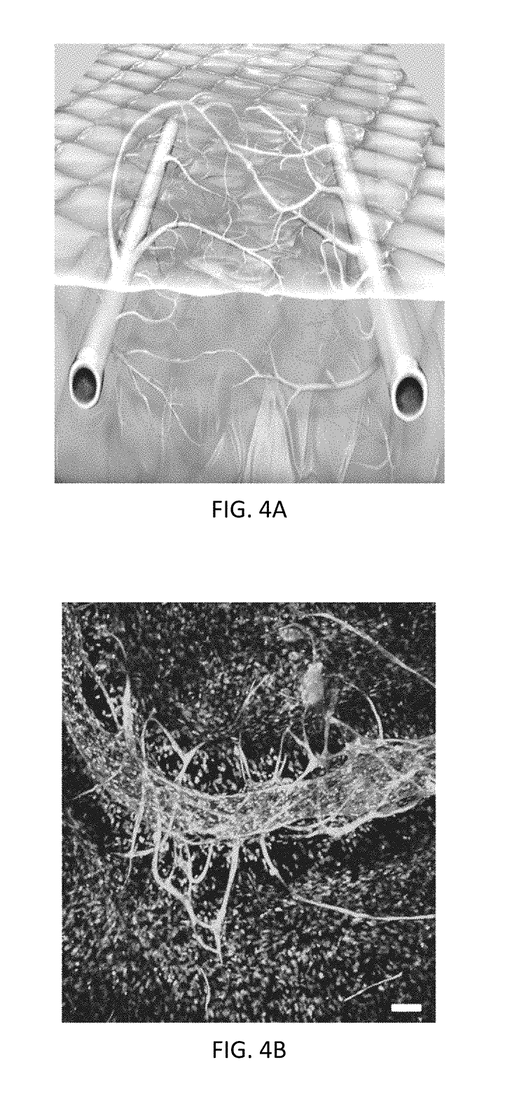

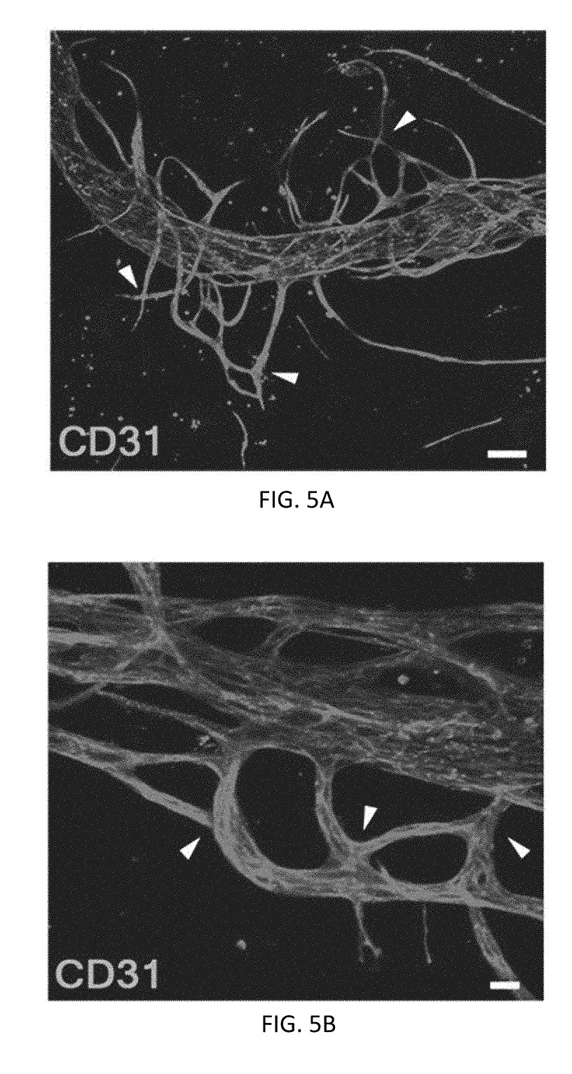

The human endothelial microvessels (HEMV) are constructed using porous, polymeric materials to generate a hollow tubule. In embodiments, the wall thereof includes one or more concentric layers of polymer, wherein the vessel has an outer diameter of between 5 and 8000 microns and wherein each individual layer of polymer has a thickness of between 0.1 and 250 microns. During construction of the tubule, cellular components such as endothelia and/or smooth muscle cells are incorporated therein to generate a cell-laden blood vessel that can recapitulate normal blood vessel function. The HEMV are easily manipulated and placement of the HEMV is unrestricted as the vessels themselves can be incorporated into nearly all dimensions and depths of a tissue model. Furthermore, experiments have established that HEMV respond to environmental cues and are capable of undergoing neovascularization, growing out from the main HEMV and allowing perfusion of the surrounding tissue (FIGS. 4B, 5A, and 5B).

Use HEMVs for vascularized skin constructs will not only allow thicker skin constructs but also better recapitulate the dynamics of human skin. To construct the VSC, keratinocytes, fibroblasts and melanocytes can be grown in a tissue construct surrounding an embedded HEMV using the device akin to the type disclosed US Patent Application Publication 2016/0130543 (incorporated by reference for this purpose) including a manifold tissue chamber.

In one embodiment, dermal fibroblasts can make up the dermal layer and act as a connective tissue support for epidermal adhesion. Human dermal fibroblast can be grown in culture, collected and resuspended, for example in in a 5% wt/vol mixture of gelatin methacrylate (GelMA) which serves as the extracellular matrix necessary for fibroblast outgrowth. The mixture of GelMA and dermal fibroblasts can be added to the manifold tissue chamber and photopolymerized to solidify, generating the dermis. Dermal fibroblasts can then be allowed to grow for a period, typically 5-10 days, after which human keratinocytes and human melanocytes (grown in a similar fashion), will can collected and layered on top of the dermis, thus constructing the epidermis. Of note, melanocytes can also be omitted from the model to generate a more basic skin construct. The keratinocyte and melanocyte mixture can be allowed to grow for a period of, for example, 5-10 days after which growth media surrounding the culture will be reduced permitting an air-liquid interface to become established around the skin. The air-liquid interface is an integral step to the protocol which triggers differentiation of the epidermal keratinocytes to generate the differentiated outer layer of the skin, called the stratum corneum. This differentiation has the keratinocytes acquiring characteristics of corneocytes can be observed by morphology and/or changes in protein expression.

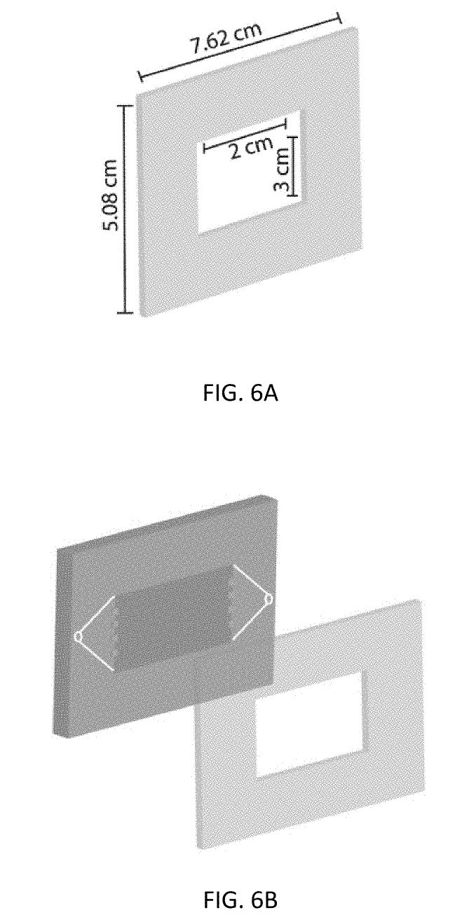

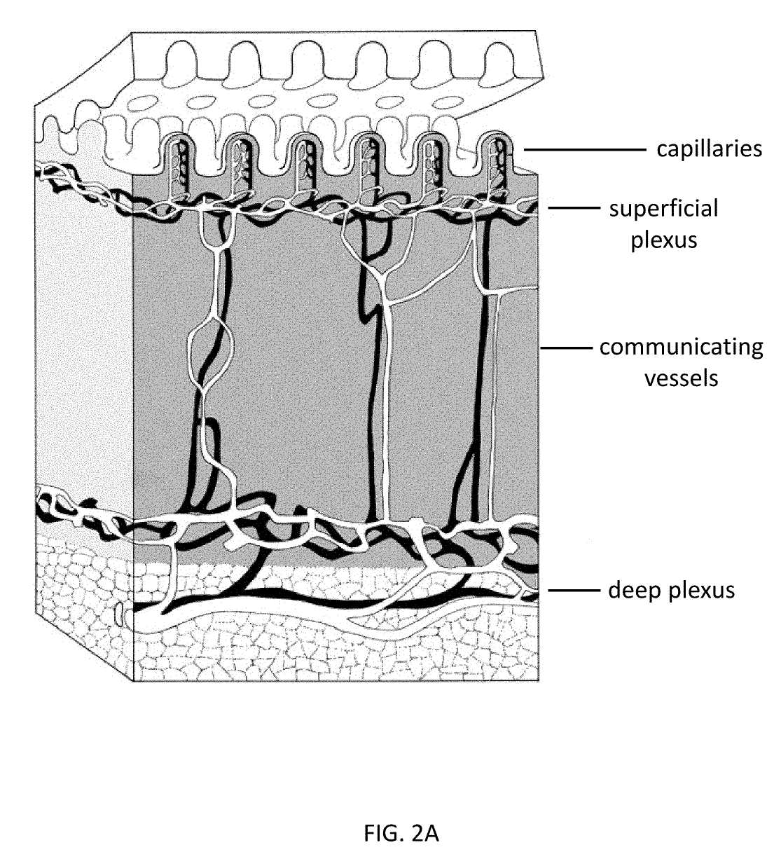

A vascular skin construct was prepared with with an embedded HEMV. During these steps growth media can be recirculated, for example using a peristaltic pump, through HEMVs serving as cellularized conduits to perfuse the VSC, thus maintaining tissue viability and removing waste. Not only do HEMV act as conduits, but they permit normal cellular functions such as engaging in cell signaling between neighboring cells, angiogenesis with generation of branched growths (endothelial sprouts), and response to shear stress during perfusion by appropriately modulating gene expression. One change difference to the original manifold device described in US Patent Application Publication 2016/0130543 involves the use of an alternative to the upper glass plate which is used for purposes of sealing the tissue manifold device. Instead, a partially exposed upper glass plate will allow an air-liquid interface to be established (FIGS. 6A and 6B) as exposure to atmospheric oxygen tis critical for epidermal keratinocyte differentiation as previously mentioned. A laser etching approach can be used to generate the plate, for example by using it to remove an inner section of a 5×7 cm glass plate exposing only the center section of the tissue construct and leave other components of the glass plate in tact so as to properly seal the VSC.

Advantages

It is possible to have both the VSC and HEMV that have cells only of human origin and eliminate complementarity issues present when using current in vivo models. Not only will this improve the compatibility, but it also provides an alternative to animal models which can be costly, subject to stringent regulation, and may be otherwise undesirable. Ultimately, the functionality of an in vitro VSC improves upon current skin models which lack any type of vasculature and are therefore limited to thin skin modeling. Using the VSC, skin transmission studies, immune-related experimentation, and other more advanced research can be accomplished. Importantly, not only can growth media be perfused through the device in order to maintain tissue survival, but other cell types beyond skin can now become incorporated into the model. For example, blood not only contains plasma proteins but also hematopoietic cells such as human red blood cells and various human-derived white blood cells such as macrophages, B- and T-cells, dendritic cells, etc. which can be incorporated in order to address immune-related activation following a given treatment, studies not possible using current skin equivalents.

Free-standing engineered blood vessels allow placement into tissue in various positions as desired, unlike other rigid in vitro tissue models with defined parameters.

Current in vitro skin models are not vascularized. This limits modeling to thin skin (<1 mm). However, many areas of the human body are >3 mm (palms of hands, soles of feet). The disclosed technique allows for better modeling of skin of greater thickness.

In vivo animal models suffer from reproducibility, species complementarity and access as bans on some studies have been in place since 2013 in the European Union.

Vascularized skin allows thick skin constructs, skin transmission studies (small molecules, pathogens, chemical agent) transmission across the skin and into the vasculature as well as long-term viability assays.

This model allows the study of transmission of various agents including compositions intended to be used as topical treatments on the skin, pathogens (including fungi, bacteria, and/or viruses) and the like through the skin. Analysis can be made of distribution to the vasculature by sampling perfused material including blood-simulants passed through the vessel(s) or growth media used in a chamber and testing for transmission/accumulation at various time points.

### CONCLUDING REMARKS

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

### REFERENCES

Adams, R. H. and K. Alitalo (2007). “Molecular regulation of angiogenesis and lymphangiogenesis.” Nat Rev Mol Cell Biol 8(6): 464-478.
Berking, C. and M. Herlyn (2001). “Human skin reconstruct models: a new application for studies of melanocyte and melanoma biology.” Histol Histopathol 16(2): 669-674.
Conway, K., D. Morgan, et al. (1992). “Tumorigenic suppression of a human cutaneous squamous cell carcinoma cell line in the nude mouse skin graft assay.” Cancer Res 52(23): 6487-6495.
Ehsan, S. M. and S. C. George (2013). “Nonsteady state oxygen transport in engineered tissue: implications for design.” Tissue Eng Part A 19(11-12): 1433-1442.
Hartung, T., B. J. Blaauboer, et al. (2011). “An expert consortium review of the EC-commissioned report “alternative (Non-Animal) methods for cosmetics testing: current status and future prospects—2010”.” ALTEX 28(3): 183-209.
Itoh, M., N. Umegaki-Arao, et al. (2013). “Generation of 3D skin equivalents fully reconstituted from human induced pluripotent stem cells (iPSCs).” PLoS One 8(10): e77673.
Juhasz, I., S. M. Albelda, et al. (1993). “Growth and invasion of human melanomas in human skin grafted to immunodeficient mice.” Am J Pathol 143(2): 528-537.
Kang, H. W, S. J. Lee, et al. (2016). “A 3D bioprinting system to produce human-scale tissue constructs with structural integrity.” Nat Biotechnol 34(3): 312-319.
Natarajan, V. T., P. Ganju, et al. (2014). “Multifaceted pathways protect human skin from UV radiation.” Nat Chem Biol 10(7): 542-551.
Paolini, C., A. Orecchia, et al. (2013). “Skin equivalents: a tool for the discovery and validation of pharmacodynamic biomarkers.” Cancer Invest 31(1): 60-66.
Prost-Squarcioni, C. (2006). “[Histology of skin and hair follicle].” Med Sci (Paris) 22(2): 131-137.
Roop, D. R., D. R. Lowy, et al. (1986). “An activated Harvey ras oncogene produces benign tumours on mouse epidermal tissue.” Nature 323(6091): 822-824.
Sonksen, J. a. C., J. (1999). “Circulation of the Skin.” Current Anaesthesia and Critical Care 10: 58-63.
Sonner, Z., E. Wilder, et al. (2015). “The microfluidics of the eccrine sweat gland, including biomarker partitioning, transport, and biosensing implications.” Biomicrofluidics 9(3): 031301.
Tian, L. and S. C. George (2011). “Biomaterials to prevascularize engineered tissues.” J Cardiovasc Transl Res 4(5): 685-698.
von Aulock, S. (2013). “EU: final ban on animal experiments for cosmetic ingredients implemented.” ALTEX 30(2): 268-269.
Worst, P. K., I. C. Mackenzie, et al. (1982). “Reformation of organized epidermal structure by transplantation of suspensions and cultures of epidermal and dermal cells.” Cell Tissue Res 225(1): 65-77.
Worst, P. K., E. A. Valentine, et al. (1974). “Formation of epidermis after reimplantation of pure primary epidermal cell cultures from perinatal mouse skin.” J Natl Cancer Inst 53(4): 1061-1064.
Yamaguchi, Y. and V. J. Hearing (2014). “Melanocytes and their diseases.” Cold Spring Harb Perspect Med 4(5).

What is claimed is:

1. A synthetic skin tissue comprising:
a synthetic blood vessel in the form of a hollow tubule comprising a porous polymer and incorporating living endothelial cells and living smooth muscle cells within the vessel;
living fibroblasts in a matrix surrounding the vessel; and
living keratinocytes disposed atop the fibroblasts, wherein an upper layer of the keratinocytes is in a differentiated state with characteristics of corneocytes.

2. The synthetic tissue of claim 1, wherein said synthetic skin tissue has a thickness of greater than one millimeter.

3. The synthetic tissue of claim 1, wherein said synthetic blood vessel has a branched morphology.

4. The synthetic tissue of claim 1, further comprising living melanocytes disposed among the keratinocytes.

5. The synthetic tissue of claim 1, wherein all of the cells are of human origin.

6. The synthetic tissue of claim 1, disposed inside a perfusion manifold device comprising an inlet port and an outlet port configured to allow fluid flow through said vessel, the device having an opening exposing the the upper layer of keratinocytes to the atmosphere.

7. The synthetic tissue of claim 1, wherein said vessel has a wall comprising one or more concentric layers of the polymer, wherein the vessel has an outer diameter of between 5 and 8000 microns and wherein each individual layer of polymer has a thickness of between 0.1 and 250 microns.

8. A vascularized skin construct comprising:
a synthetic skin tissue comprising a synthetic blood vessel in the form of a hollow tubule comprising a porous polymer and incorporating living human endothelial cells and living smooth muscle cells within the vessel, living human fibroblasts in a matrix surrounding the vessel, and living human keratinocytes disposed atop the fibroblasts, wherein an upper layer of the keratinocytes is in a differentiated state with characteristics of corneocytes; and
a perfusion manifold device comprising an inlet port and an outlet port configured to allow fluid flow through the vessel, the device having an opening exposing the the upper layer of keratinocytes to the atmosphere.

9. The vascularized skin construct of claim 8, wherein said synthetic skin tissue has a thickness of greater than one millimeter.

10. The vascularized skin construct of claim 8, wherein said synthetic blood vessel has a branched morphology.

11. The synthetic tissue of claim 8, further comprising living human melanocytes disposed among the keratinocytes.

12. The synthetic tissue of claim 8, wherein said vessel has a wall comprising one or more concentric layers of the polymer, wherein the vessel has an outer diameter of between 5 and 8000 microns and wherein each individual layer of polymer has a thickness of between 0.1 and 250 microns.
